(19) United States
(12) Patent Application Publication
(10) Pub. No.: US 2003/0187378 A1
(43) Pub. Date: Oct. 2, 2003

(54) Medical padding product with adjustable and removable gel pad

(76) Inventors: Robert Scott Gaylord, Matthews, NC (US); Eric Lee Gaylord, Matthews, NC (US)

Correspondence Address: W. Thad Adams, III, Adams, Schwartz & Evans, P.A., 2180 Two Wachovia Center, Charlotte, NC, 28282, US

(21) Appl. No.: 10/114,818

(22) Filed: Apr. 2, 2002

(51) Int. Cl. A61F 5/32; A61F 5/34

(52) U.S. Cl. 602/62; 602/75; 606/204

(57) ABSTRACT

A medical padding product such as a wrist splint or support, for providing a protective pad to a desired body area to be protected, and including a support base for being applied to a site overlying and surrounding the body area to be protected. A gel pad is adjustably and releasably positioned on the base to overlie the body area to be protected. Pad attaching elements, for example hook or loop material, is carried by the gel pad on one major surface thereof for releasably attaching to a complementary hook or loop material carried on the surface of the base. According to one preferred embodiment of the invention, the cushioning pad is a gel pad.

### TECHNICAL FIELD AND BACKGROUND OF THE INVENTION

This invention relates to a medical padding product of the type used to support an injured or wounded area of the body, particularly an extremity such as the arms, legs, hands and feet areas. For purposes of illustration in this application, the invention is described with reference to a wrist support, that may be used to support a wrist during recovery from a sprain or break, to ameliorate conditions such as carpel tunnel syndrome, or to immobilize the wrist after surgery. There are, of course, many types and brands of medical padding products intended to protect and/or immobilize a particular part of the body. The protection may be for the purpose of preventing injury, or for protecting an injury from further damage during healing. Many of these products are sold in different sizes, while other products provide a “one size fits all” configuration that, in reality, fit few individuals exactly as needed for best performance.

The present invention provides a medical padding product that utilizes a conventional type of base, but that incorporates a gel pad that can be easily positioned and repositioned as needed by the patient. The wrist support product permits the wrist to be supported during recovery from a number of different conditions, with precise adjustment possible as frequently as desired and within a broad range.

### SUMMARY OF THE INVENTION

Therefore, it is an object of the invention to provide a medical padding product that is easy to apply.

It is another object of the invention to provide a medical padding product that is adjustable on the body part to be protected.

It is another object of the invention to provide a medical padding product that has an adjustable gel pad that can be easily and quickly positioned to achieve an exactly proper fit.

It is another object of the invention to provide a medical padding product that has an adjustable gel pad that can be easily and quickly repositioned as needed to accommodate decreased edema as healing progresses.

It is another object of the invention to provide a medical padding product has an adjustable gel pad that can be cut to a particular desired shape and positioned as needed to accommodate decreased edema as healing progresses.

It is another object of the invention to provide a wrist support which achieves the above-identified objects.

These and other objects of the present invention are achieved in the preferred embodiments disclosed below by providing a medical padding product for providing a protective pad to a desired body area to be protected, and comprising a support base for being applied to a site overlying and surrounding the body area to be protected, a gel pad adjustably and releasably positioned on the base to overlie the body area to be protected, and pad attachment means carried by the gel pad on one major surface thereof for releasably attaching to a complementary pad attachment means carried on the surface of the base.

According to one preferred embodiment of the invention, the cushioning pad comprises a gel pad having fixed to one surface thereof pad attachment means for releasably attaching to a complementary pad attachment means carried on the surface of the base.

According to another preferred embodiment of the invention, the pad attachment means comprises a multiplicity of flexible hooks and the complementary pad attachment means comprises a fabric having a mass of fibrous loops on the surface thereof for being releasably entangled on the hooks.

According to yet another preferred embodiment of the invention, the pad attachment means and the complementary pad attachment means comprise complementary touch fastener elements.

According to yet another preferred embodiment of the invention, the securing means comprise complementary touch fastener elements.

According to yet another preferred embodiment of the invention, the gel pad comprises a solid viscoelastic gel.

According to yet another preferred embodiment of the invention, the gel pad comprises a polymer gel impregnated with mineral oil.

According to yet another preferred embodiment of the invention, the support base comprises an elongate synthetic elastic fabric having a dense mass of loops on an outwardly-facing side, a smooth fabric surface on a reverse, body-contacting side, and a cushion padding sandwiched between the outwardly-facing side and body-contacting side.

According to yet another preferred embodiment of the invention, a wrist support is provided, and comprises a support base for being extended around the wrist and adjacent areas of the back and palm of the hand and encircling and supporting the medial and lateral aspects of the wrist in a stationary neutral position relative to the lower arm and hand. Securing means are provided for securing the base around the wrist and between the thumb and forefinger for securing the base around the back and palm of the hand. A gel pad is provided of a suitable size and shape to extend along and conform to the medial aspect of the wrist and adjacent palm area of the hand. Pad attachment means are carried by the gel pad on one major surface thereof for releasably attaching to a complementary pad attachment means carried on the surface of the base.

According to yet another preferred embodiment of the invention, the gel pad of the wrist support has a length at least twice its width and at least 80 percent of the width of the base.

According to yet another preferred embodiment of the invention, the pad attachment means of the wrist support comprises a multiplicity of flexible hooks and the complementary pad attachment means comprises a fabric having a mass of fibrous loops on the surface thereof for being releasably entangled on the hooks.

According to yet another preferred embodiment of the invention, the pad attachment means and the complementary pad attachment means of the wrist support comprise complementary touch fastener elements.

According to yet another preferred embodiment of the invention, the securing means of the wrist support comprise complementary touch fastener elements.

According to yet another preferred embodiment of the invention, the gel pad of the wrist support comprises a solid viscoelastic gel.

According to yet another preferred embodiment of the invention, the gel pad of the wrist support comprises a polymer gel impregnated with mineral oil.

According to yet another preferred embodiment of the invention, the support base of the wrist support comprises a synthetic elastic fabric having a dense mass of loops on an outwardly-facing side, a smooth fabric surface on a reverse, hand-contacting side, and a cushion padding sandwiched between the outwardly-facing side and hand-contacting side.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 is a plan view of the inner side of a wrist support according to one embodiment of the invention;

FIG. 2 is a plan view of the outer side of the wrist support shown in FIG. 1;

FIG. 3 is a plan view according to FIG. 1, and showing the gel pad and the means by which is can be removed and repositioned;

FIG. 4 is a plan view of the gel pad shown in FIGS. 1 and 3;

FIG. 5 is a cross-section taken along lines 5--5 of FIG. 4;

FIG. 6 is a view of the medial aspect of the hand, wrist and arm with the wrist support properly positioned thereon; and

FIG. 7 is a view of the lateral aspect of the hand, wrist and arm with the wrist support properly positioned thereon.

### DESCRIPTION OF THE PREFERRED EMBODIMENT AND BEST MODE

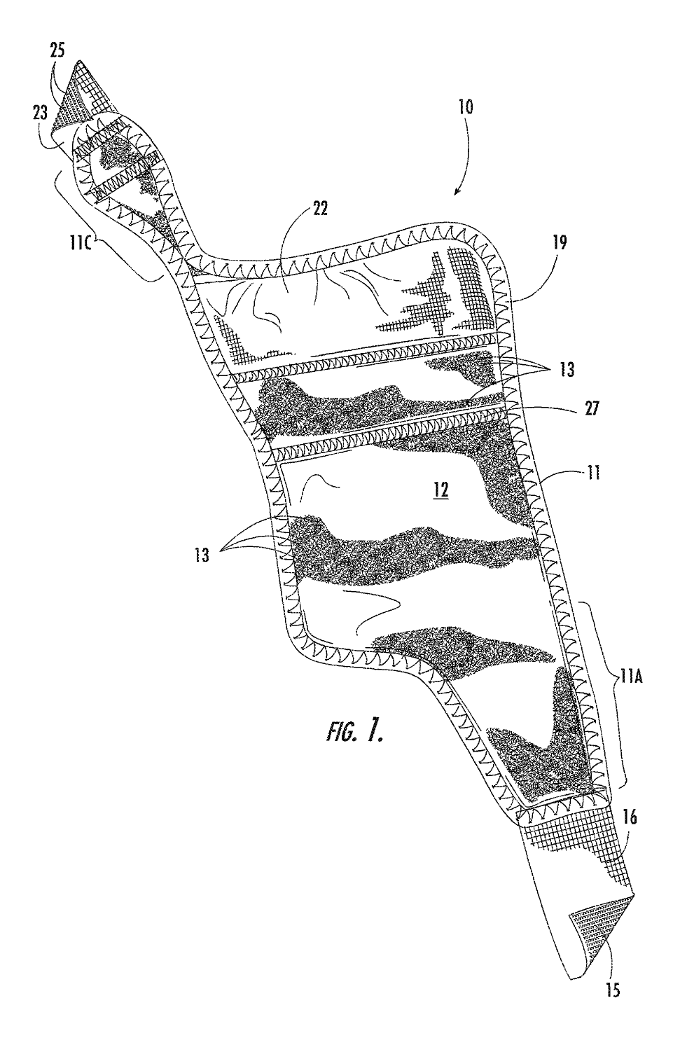

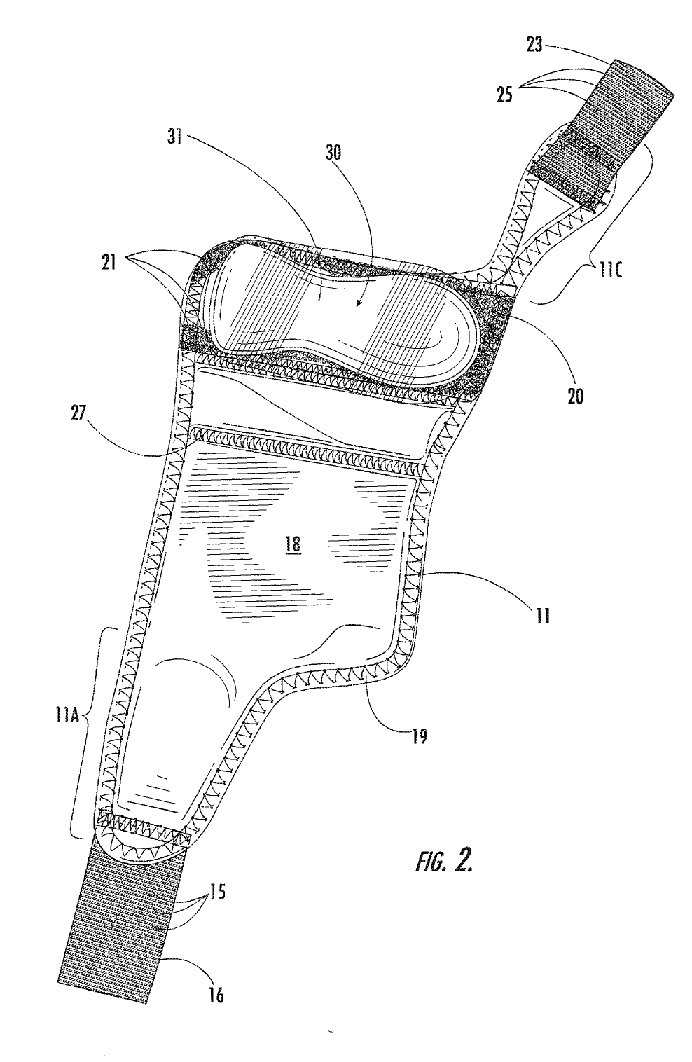

Referring now specifically to the drawings, a medical padding product according to the present invention is illustrated in FIGS. 1 and 2, and shown generally at reference numeral 10. The particular embodiment of the medical padding product is a wrist support, which term is used below.

The wrist support 10 is fabricated of a support base 11 shaped to extend and around and encircle the lower arm, wrist and adjacent hand areas. The base 11 is preferably fabricated of a laminated fabric having a outer surface 12 comprised of a multitude of uncut fibrous loops 13, as shown in FIG. 1. The loops 13 mate with hooks 15 carried on one side of an attachment tape 16 sewn onto an integrally-formed strap 11A on one end of the base 11. The inner surface 17 of the base 11 is a smooth, knitted fabric 18 intended to provide a comfortable, low friction structure to reside next to the skin of the wearer. The fabric 18 includes an amount of spandex material sufficient to provide a degree of elasticity to the wrist support 10.

A thin layer of a breathable foam, not shown, is sandwiched between the inner surface 12 and outer surface 18 to provide further comfort. This laminated material is generally referred to as “neoprene replacement fabric” and is sold by several suppliers. The entire support base 11 is bound by a binding tape 19 sewn over and enclosing the raw edges with zig-zag stitches.

The wrist support also includes a pad attachment strip 20 having loops 21 on the inner surface, FIG. 2, and a nylon webbing material 22 on the outer surface, FIG. 1. The webbing 22 provides an abrasion resistant area in the palm of the hand which increases durability.

A strap 11C integrally formed with the base 11 extends between the thumb and forefinger and permits the forward end of the support 10 to be secured to the hand. An attachment tape 23 having hooks 25 on one side mates with the loops 13 on the outer surface 12 of the base 11. A transverse seam 27 add dimensional stability to the base 11.

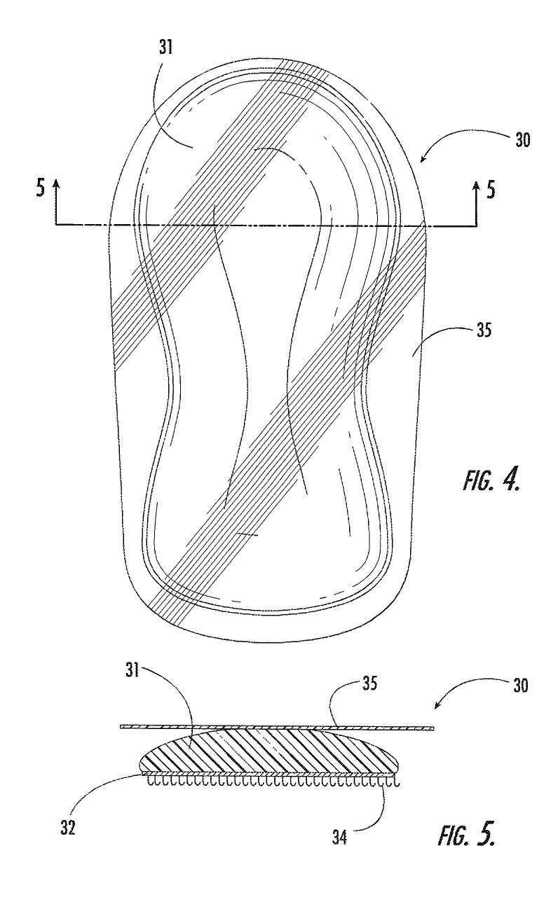

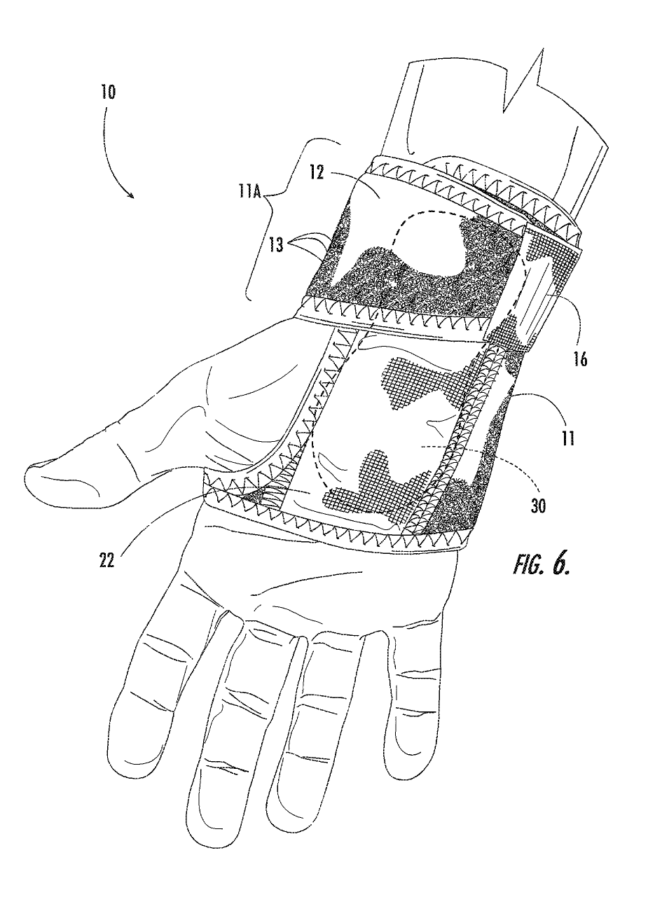

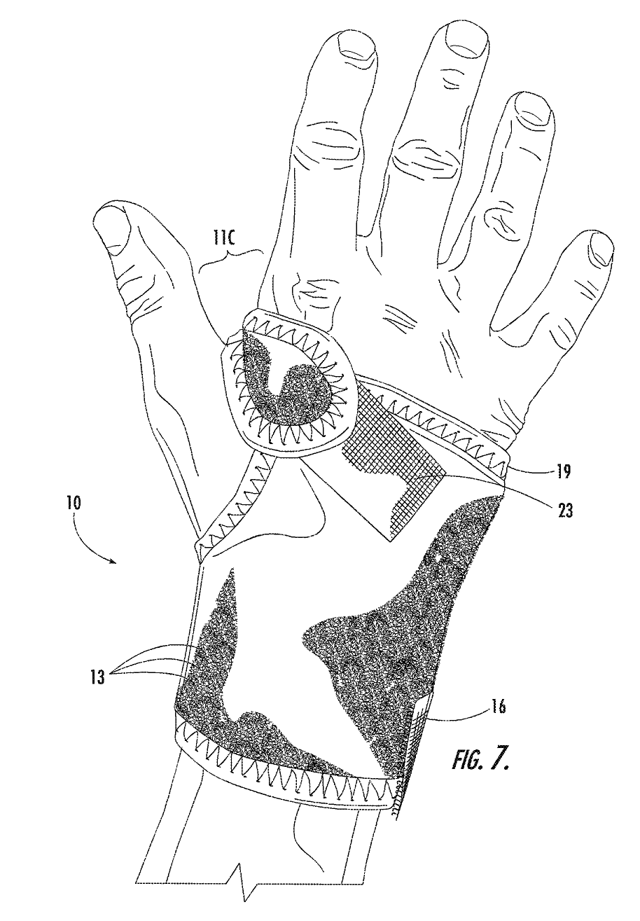

Referring now to FIGS. 2-5, a gel pad assembly 30 is provided, which includes as its principal component an elongate pad 31 fabricated of a polymer gel material. One supplier of a suitable material is Silipos, Inc., that markets a product known as a carpel gel pad under the product designation No. 14205. This product includes mineral oil that acts as a lubricating agent to help reduce skin sensitivity, flatten scar tissue and increase range of motion. The jelly-like compressibility of the pad also insulates against shock and vibration. Other products having comparable characteristics are also usable. The gel pads are furnished by the manufacturer with an contact adhesive-coated fabric adhered to one face. To this fabric is adhered a tape 32 having an adhesive on the inner side. The outwardly-facing side of the tape 32 is covered with hooks 34 that mate with the complementary loops 21 on the inner surface of the support 10.

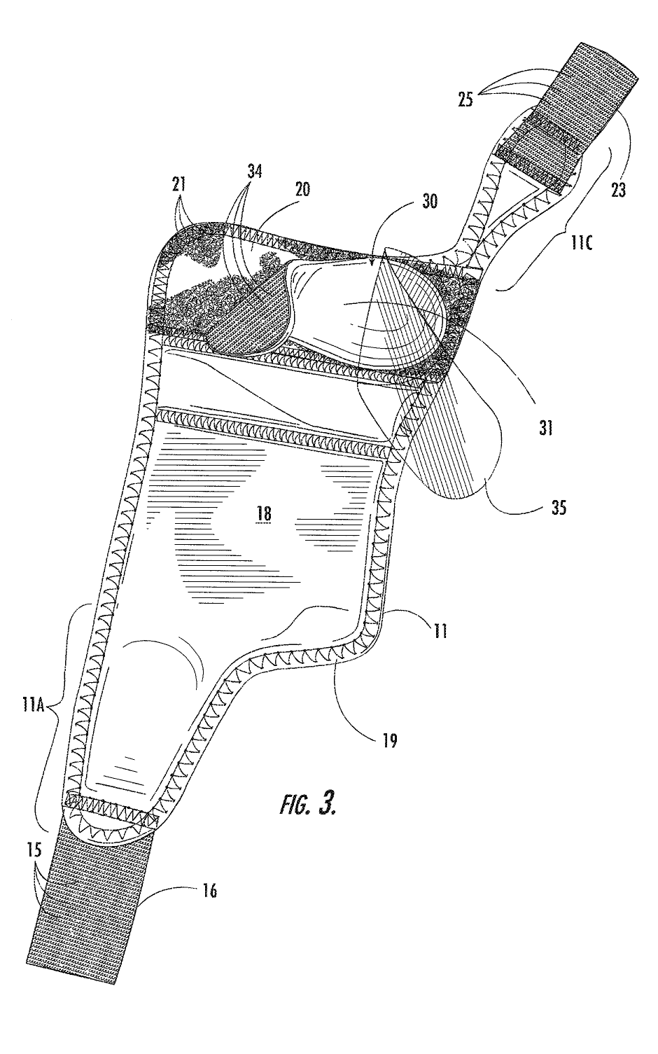

The gel pad 31 is covered by a protective film 35 which 0.6 is disposed of immediately prior to use. As can best be seen with reference to FIG. 3, the gel pad assembly 30 is easily removable by grasping one end of the gel pad 31 and peeling it away from the underlying loops 21 to which the gel pad assembly 30 is attached. The gel pad assembly 30 may be partially or completely detached and repositioned, or can be removed and replaced with a new gel pad assembly 30.

The gel pad 31 is preferably 4.5 inches (11.5 mm) long and 2.5 inches (5 cm) wide at its widest points and 1.5 inches (4 cm) wide in the narrowest point. According to the preferred embodiment disclosed herein, the gel pad 31 is preferably ⅜inches (0.9 cm) thick.

It is believed that the gel pad 31 and the hook and loop materials disclosed represent the preferred means of constructing this product. However, other padding materials, for example, open or closed cell synthetic or rubber foam, silicone and other synthetic padding materials may be used. Similarly, the use of straps carrying buckles, such as D-ring buckles may also be used. Likewise, there are several materials, such as neoprene, canvas, knitted synthetic fabrics, either laminated or unlaminated, which may be used as the base material.

A medical padding product is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

I claim:

1. A medical padding product for providing a protective pad to a desired body area to be protected, and comprising:
(a) a support base for being applied to a site overlying and surrounding the body area to be protected;
(b) a gel pad adjustably and releasably positioned on the base to overlie the body area to be protected; and
(c) pad attachment means carried by the gel pad on one major surface thereof for releasably attaching to a complementary pad attachment means carried on the surface of the base.

2. A medical padding product according to claim 1, wherein said cushioning pad comprises a gel pad having fixed to one surface thereof pad attachment means for releasably attaching to a complementary pad attachment means carried on the surface of the base.

3. A medical padding product according to claim 2, wherein the pad attachment means comprises a multiplicity of flexible hooks and said complementary pad attachment means comprises a fabric having a mass of fibrous loops on the surface thereof for being releasably entangled on the hooks.

4. A medical padding product according to claim 2, wherein the pad attachment means and the complementary pad attachment means comprise complementary touch fastener elements.

5. A medical padding product according to claim 1, 2, 3 or 4, wherein said securing means comprise complementary touch fastener elements.

6. A medical padding product according to claim 1, 2, 3 or 4, wherein said gel pad comprises a solid viscoelastic gel.

7. A medical padding product according to claim 6, wherein the gel pad comprises a polymer gel impregnated with mineral oil.

8. A medical padding product according to claim 6, wherein the support base comprises an elongate synthetic elastic fabric having a dense mass of loops on an outwardly-facing side, a smooth fabric surface on a reverse, body-contacting side, and a cushion padding sandwiched between the outwardly-facing side and body-contacting side.

9. A wrist support, comprising:
(a) a support base for being extended around the wrist and adjacent areas of the back and palm of the hand and encircling and supporting the medial and lateral aspects of the wrist in a stationary neutral position relative to the lower arm and hand;
(b) securing means for securing the base around the wrist and between the thumb and forefinger for securing the base around the back and palm of the hand;
(c) a gel pad of a suitable size and shape to extend along and conform to the medial aspect of the wrist and adjacent palm area of the hand; and
(d) pad attachment means carried by the gel pad on one major surface thereof for releasably attaching to a complementary pad attachment means carried on the surface of the base.

10. A wrist support according to claim 9, wherein said gel pad has a length at least twice its width and at least 80 percent of the width of the base.

11. A wrist support according to claim 9, wherein the pad attachment means comprises a multiplicity of flexible hooks and said complementary pad attachment means comprises a fabric having a mass of fibrous loops on the surface thereof for being releasably entangled on the hooks.

12. A wrist support according to claim 9, wherein the pad attachment means and the complementary pad attachment means comprise complementary touch fastener elements.

13. A wrist support according to claim 9, 10, 11 or 12, wherein said securing means comprise complementary touch fastener elements.

14. A wrist support according to claim 9, 10, 11 or 12, wherein said gel pad comprises a solid viscoelastic gel.

15. A wrist support according to claim 14, wherein the gel pad comprises a polymer gel impregnated with mineral oil.

16. A wrist support according to claim 9, wherein the support base comprises a synthetic elastic fabric having a dense mass of loops on an outwardly-facing side, a smooth fabric surface on a reverse, hand-contacting side, and a cushion padding sandwiched between the outwardly-facing side and hand-contacting side.
